(19) United States
(12) Patent Application Publication
(10) Pub. No.: US 2013/0209393 A1
(43) Pub. Date: Aug. 15, 2013

(54) THERAPEUTIC PEPTIDE COMPOSITIONS AND METHODS

(71) Applicants: Bassam B. Damaj, San Diego, CA (US); Richard Martin, San Diego, CA (US)

(72) Inventors: Bassam B. Damaj, San Diego, CA (US); Richard Martin, San Diego, CA (US)

(73) Assignee: NEXMED HOLDINGS, INC., San Diego, CA (US)

(21) Appl. No.: 13/703,003

(22) PCT Filed: May 4, 2011
(86) PCT No.: PCT/US11/00790

§ 371 Date: Feb. 28, 2013

Related U.S. Application Data
(60) Provisional application No. 61/343,815, filed on May 4, 2010.

(51) Int. Cl. A61K 47/18

(52) U.S. Cl. CPC A61K 47/18
USPC 424/85.1; 514/5.9; 424/141.1; 424/133.1; 514/11.7

(57) ABSTRACT

Therapeutic peptide compositions comprise a therapeutic peptide, such as insulin, together with an alkyl N,N-disubstituted amino acetate, such as dodecyl 2-(N,N-dimethylamino) propionate.

### CROSS-REFERENCE TO RELATED APPLICATION

This application claims the priority of U.S. Provisional Application Ser. No. 61/343,815, filed May 4, 2010, the disclosures of which are incorporated in their entirety herein by reference.

### FIELD OF INVENTION

This invention relates to therapeutic peptide compositions and methods of delivery for such compositions.

### BACKGROUND OF INVENTION

Peptides are mediators of biological functions. The unique intrinsic properties of peptides make peptides attractive therapeutic agents because peptides exhibit relatively high biological activity and specificity as well as relatively low toxicity. Therapeutic peptides in vivo have drawbacks, however, such as relatively low stability, susceptibility to enzymatic degradation, relatively poor tumor penetration in cancer treatments, to name a few.

While therapeutic peptides are viable alternatives to other biopharmaceuticals, their stability and half-life in vivo continues to be a concern.

### SUMMARY OF INVENTION

The present therapeutic peptide compositions embodying the present invention provide a depot-like effect when administered to a patient, thereby extending many fold the therapeutic duration of the administered peptide.

The therapeutic peptide compositions of the present invention comprise the therapeutic peptide and an alkyl N,N-disubstituted amino acetate, if desired, together with a physiologically acceptable carrier. The dosage and dosage form of the therapeutic peptide in any given case depends on the condition being treated, the particular therapeutic peptide that is used to treat the condition, as well as the desired route of administration.

A composition comprising insulin and dodecyl 2-(N,N-dimethylamino) propionate hydrochloride is particularly well suited for controlling blood glucose levels in diabetic patients.

### BRIEF DESCRIPTION OF DRAWINGS

In the drawings,

FIG. 1 is a graph showing blood glucose levels in normal mice over a period of time after administration of insulin in saline and in dodecyl 2-(N,N-dimethylamino) propionate;

FIG. 2 is a graph of blood serum levels of Biot-Rituxan versus time in samples from hamsters that received subcutaneous doses in saline and in dodecyl 2-(N,N-dimethylamino) propionate;

FIG. 3 is a graph of blood plasma levels of liraglutide versus time in samples from mice that received subcutaneous doses in a clinical formulation with and without dodecyl 2-(N,N-dimethylamino) propionate present;

FIG. 4 is a graph showing blood glucose levels in mice receiving 2.5 IU/kg of insulin subcutaneously in 0.9% saline and 20% DDAIP.HCl (pH 8.5) in water over an 8-hour period; and

FIG. 5 is a graph showing blood glucose levels in mice receiving 2.5 IU/kg of insulin subcutaneously in 0.9% saline and 20% DDAIP.HCl (pH 8.5) in water over a 26-hour period, the mice having access to food at 8 hours after injection.

### DESCRIPTION OF PREFERRED EMBODIMENTS

### Definition of Terms

The term “peptide,” as used herein and in the appended claims, refers to any compound containing two or more amino acid residues joined by an amide bond formed from the carboxyl group of one amino acid residue and the amino group of the adjacent amino acid residue. The amino acid residues may have the L-form as well as the D-form, and may be naturally occurring or synthetic, linear as well as cyclic. Also included within the term “peptide” as used herein and in the claims are polypeptides and peptide dimers which can be peptides linked C-terminus to N-terminus (tandem repeats) or peptides linked C-terminus to C-terminus (parallel repeats).

The term “therapeutic peptide,” as used herein and in the appended claims denotes a bioactive peptide that has therapeutic utility. Illustrative categories of therapeutic peptides suitable for practicing the present invention are hormones, monoclonal antibodies, extracellular matrix (ECM) peptides, enzymes, cytokines and the like.

Physiologically Acceptable Carrier: As used herein, the term “physiologically acceptable carrier” refers to a diluent, adjuvant, excipient, or the like vehicle with which a therapeutic peptide is administered. Such carriers can be sterile liquids, such as water and oils, including those of petroleum, animal, vegetable, or synthetic origin, such as peanut oil, soybean oil, mineral oil, sesame oil, tocopherols and the like, polyethylene glycols, glycerine, propylene glycol, or other synthetic solvents. Water is a preferred carrier when a therapeutic peptide is administered intravenously. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, or any compound found in the Handbook of Pharmaceutical Excipients (4^{th} edition, Pharmaceutical Press) and the like. A minor amount of wetting or emulsifying agents, or pH buffering agents such as acetates, citrates, or phosphates may also be present. Also, antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may be present.

Therapeutically Effective Amount: As used herein, the term “therapeutically effective amount” refers to those amounts that, when administered to a particular subject in view of the nature and severity of that subject's disease or condition, will have a desired therapeutic effect, e.g. an amount which will cure, prevent, inhibit, or at least partially arrest or partially prevent a target disease or condition.

Illustrative hormones are the insulins, e.g., human insulin, bovine insulin, porcine insulin, biosynthetic human insulin (Humulin®) etc., somatostatin, vasopressin, calcitonin, estrogen, progestin, testosterone, glucagon, glucagon-like peptide (GLP-1) and its analogs, e.g., liraglutide (VICTOZA®), and the like.

The monoclonal antibodies can be of various types, such as mouse, chimera, humanized, or human. Illustrative chimera-type monoclonal antibodies are Rituximab (RITUXAN®), Cetuximab (ERBITUX®), Infliximab (REMICADE®), Basiliximab (SIMULECT®), and the like. Illustrative humanized monoclonal antibodies are Trastuzumab (HERCEPTIN®), Palivizumab (SYNAGIS®), Efalizumab (RAPTIVA®), and the like. Illustrative human monoclonal antibodies are Adalimumab (HUMIRA®), Panitumumab (VECTIBIX®), and the like.

Illustrative ECM peptides are fibronectin, vitronectin, tenascin, and the like.

Illustrative enzymes are glucocerebrosidase, rhDNase, hyaluronidase, urokinase, alpha galactosidase, beta galactosidase, and the like.

Illustrative cytokines are the α, β, and γ interferons, the lymphokines, e.g., interleukin-2, interleukin-6, etc., human granulocyte colony-stimulating factor (G-CSF), e.g., filgrastim, granulocyte macrophage colony-stimulating factor (GM-CSF), recombinant, e.g., molgramostim, sargramostim (LEUKINE®), and the like.

The alkyl N,N-disubstituted amino acetates suitable for present purposes are represented by the formula

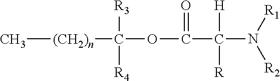

wherein n is an integer having a value in the range of about 4 to about 18; R is a member of the group consisting of hydrogen, C₁ to C₇ alkyl, benzyl and phenyl; R₁ and R₂ are members of the group consisting of hydrogen and C₁ to C₇ alkyl; and R₃ and R₄ are members of the group consisting of hydrogen, methyl and ethyl.

Preferred alkyl(N,N-disubstituted amino)-acetates are C₄ to C₁₈ alkyl(N,N-disubstituted amino)-acetates and C₄ to C₁₈ alkyl(N,N-disubstituted amino)-propionates as well as pharmaceutically acceptable salts and derivatives thereof. Exemplary specific alkyl-2-(N,N-disubstituted amino)-acetates include dodecyl 2-(N,N dimethylamino)-propionate (DDAIP):

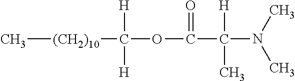

and dodecyl 2-(N,N-dimethylamino)-acetate (DDAA):

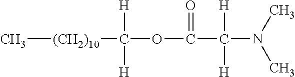

Alkyl-2-(N,N-disubstituted amino)-acetates are known. For example, dodecyl 2-(N,N-dimethylamino)-propionate (DDAIP) is available from Steroids, Ltd. (Chicago, Ill.). In addition, alkyl-2-(N,N-disubstituted amino)-alkanoates can be synthesized from more readily available compounds as described in U.S. Pat. No. 4,980,378 to Wong et al., which is incorporated herein by reference to the extent that it is not inconsistent. As described therein, alkyl-2-(N,N-disubstituted amino)-acetates are readily prepared via a two-step synthesis. In the first step, long chain alkyl chloroacetates are prepared by reaction of the corresponding long chain alkanols with chloromethyl chloroformate or the like in the presence of an appropriate base such as triethylamine, typically in a suitable solvent such as chloroform. The reaction can be depicted as follows:

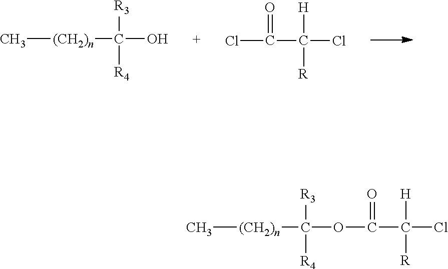

wherein n, R, R₁, R₂, R₃ and R₄ are defined as above. The reaction temperature may be selected from about 10 degrees Celsius to about 200 degrees Celsius or reflux, with room temperature being preferred. The use of a solvent is optional. If a solvent is used, a wide variety of organic solvents may be selected. Choice of a base is likewise not critical. Preferred bases include tertiary amines such as triethylamine, pyridine and the like. Reaction time generally extends from about one hour to three days.

In the second step, the long chain alkyl chloroacetate is condensed with an appropriate amine according to the scheme:

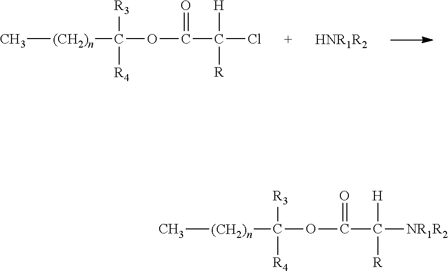

wherein n, R, R₁, R₂, R₃ and R₄ are defined as before. Excess amine reactant is typically used as the base and the reaction is conveniently conducted in a suitable solvent such as ether. This second step is preferably run at room temperature, although temperature may vary. Reaction time usually varies from about one hour to several days. Conventional purification techniques can be applied to ready the resulting ester for use in a pharmaceutical compound.

The amount of alkyl N,N-disubstituted aminoacetate, such as DDAIP, present in the therapeutic peptide compositions can vary, and depends in part on the particular peptide to be administered as well as the route of administration.

This invention is further illustrated by the following examples.

### Example 1

### Delivery of Insulin in DDAIP

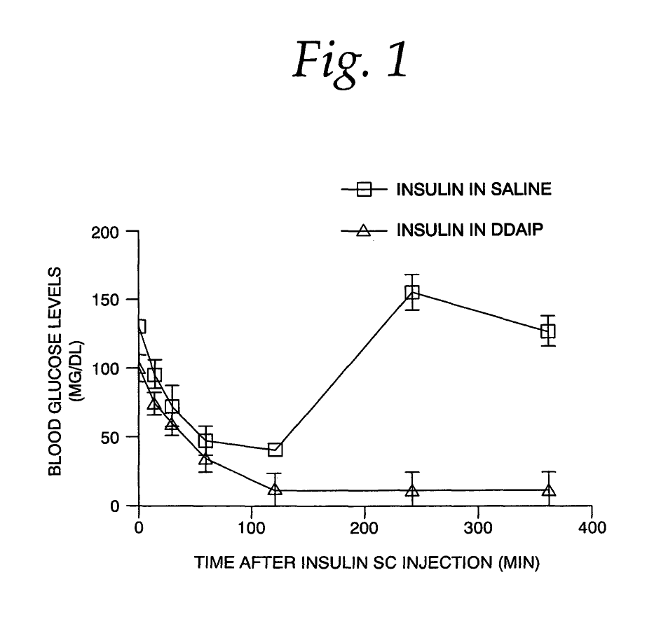

Insulin (Bovine insulin, Sigma) at a concentration of 3.2 IU/ml) was administered subcutaneously (single dose, 0.4 IU/mouse) to normal mice, in DDAIP (free base) (n=3) and in phosphate buffered saline (n=2) The blood glucose levels after injection were monitored before administration and at various time intervals thereafter. The results are shown in FIG. 1 and indicate that DDAIP has a depot like releasing effect on the therapeutic peptide, such as insulin, delivered subcutaneously.

### Example 2

### Delivery of Monoclonal Antibody in DDAIP

Formulations of biotinylated Rituximab (Biot-RITUXAN®) (9.4 mg/mL), DDAIP free base (either 4.9 or 369% w/v) and polyoxyethylene (20) sorbitan monolaurate (Tween® 20) (5% w/v) in 0.1M phosphate buffer at pH of either 5.5 or 7.4 were prepared and administered to groups of three hamsters subcutaneously (SC) as a single dose. The administered doses were 10 mg/kg subcutaneously. Another group of three hamsters received subcutaneously 10 mg/kg of Biot-RITUXAN® in saline.

Samples of blood (100 ml) were collected from the animals in red serum tubes kept on ice. The collected samples were processed by centrifugation at about 3,000 RPM for about ten minutes. The obtained supernatant serum was transferred to polypropylene tubes, placed into dry ice to freeze and then stored at minus 80° C. until analyzed. The cell fraction obtained by centrifugation was discarded.

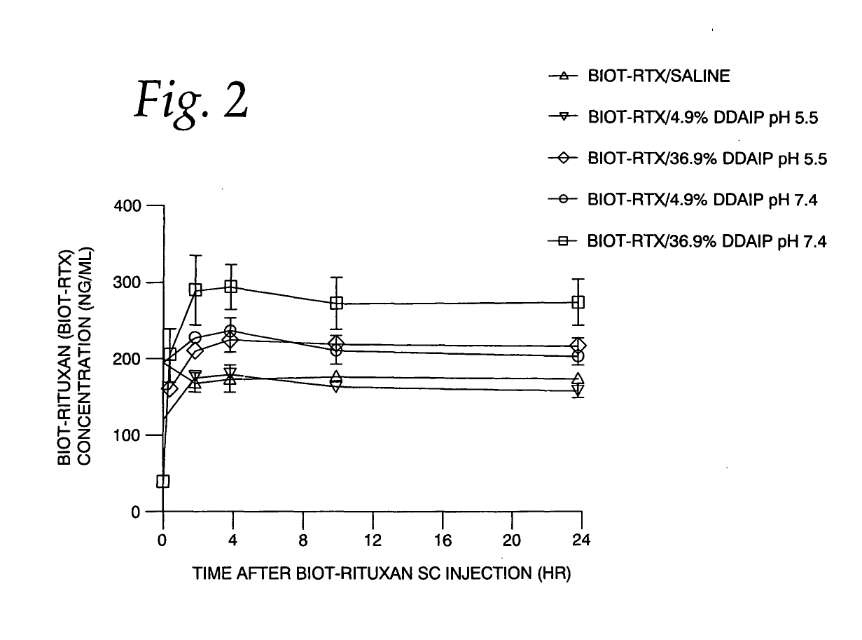

The obtained data are presented in FIG. 2. The formulations at a pH of about 7.4 provided an increase in area under the curve (AUC) of about 21% for the composition containing about 4.9 percent by weight DDAIP and about 46% for the composition containing 36.9 percent by weight of DDAIP as compared to Biot-RITUXAN® in saline.

### Example 3

### Delivery of Glucagon-like Peptide 1 (GLP-1) Analog in DDAIP.HCl

Male ICR(CD-1) mice were obtained from Harlan, USA. The mice were approximately seven weeks old, had an average weight of about 36 grams, and were fed food and water ad libitum. The experimental groupings are shown in Table I, below.

Liraglutide (VICTOZA®) was combined with 5% w/v DDAIP hydrochloride (DDAIP.HCl) and the resulting clinical formulation was administered immediately after preparation to a group of 21 mice subcutaneously (SC) as a single dose. The administered doses were 600 micrograms (μg)/mouse in 100 microliters (μl) subcutaneous. Another group of 21 mice received subcutaneously a clinical formulation of liraglutide without DDAIP.HCl present. A group of three mice received no treatment and were used as baseline controls.

TABLE I
Experimental Grouping.

| | | Dose | | Volume | | | | Bleeds |
|---|---|---|---|---|---|---|---|---|
| Mouse | | per | | per | | | | per time |
| Group | Treatment | Mouse | | Mouse | | Route | Regimen | point |
| 1 | Liraglutide | 600 | μg | 100 | μl | SC | Once | n = 3 |
| (n = 21) | (Victoza ®) | | | | | | | Note (a) |
| | Clinical Formulation | | | | | | | |
| | Note (b) | | | | | | | |
| 2 | Liraglutide | 600 | μg | 100 | μl | SC | Once | n = 3 |

| | | | | | | |
|---|---|---|---|---|---|---|
| (n = 21) | (Victoza ®) | | | | | Note (a) |
| | Clinical Formulation + 5% | | | | | |
| | w/v DDAIP · HCI | | | | | |
| 3 | None | N/A | N/A | N/A | N/A | n = 3 |
| (n = 3) | (Baseline Control) | | | | | Time = 0 |

Note (a) to Table I:
Time points were 15 min., 30 min., 1 hr., 2 hr., 4 hr., 12 hr., and 24 hr. post-dosing .
Note (b) to Table I:
Each 1 milliliter of the clinical formulation contains 6 mg of liraglutide. Each pre-filled pen contains 3 ml of solution containing 18 milligrams of liraglutide (free-base anhydrous) and the following inactive ingredient: 1.42 milligrams disodium phosphate dihydrate, 14 milligrams propylene glycol, 5.5 milligrams phenol and water q.s. for injection.

### Blood Collection:

At each time point shown in Table I, groups of mice (n=3 per data point) were anesthetized with isoflurane prior to a terminal cardiac bleed being performed with a 25 gauge needle. Blood samples were collected into K₂EDTA tubes and centrifuged at 10,000 rpm and a temperature of 4° C. for 10 minutes. Plasma samples were collected and stored at minus 80° C. until analyzed by LCMS-MS.

### Results and Conclusion:

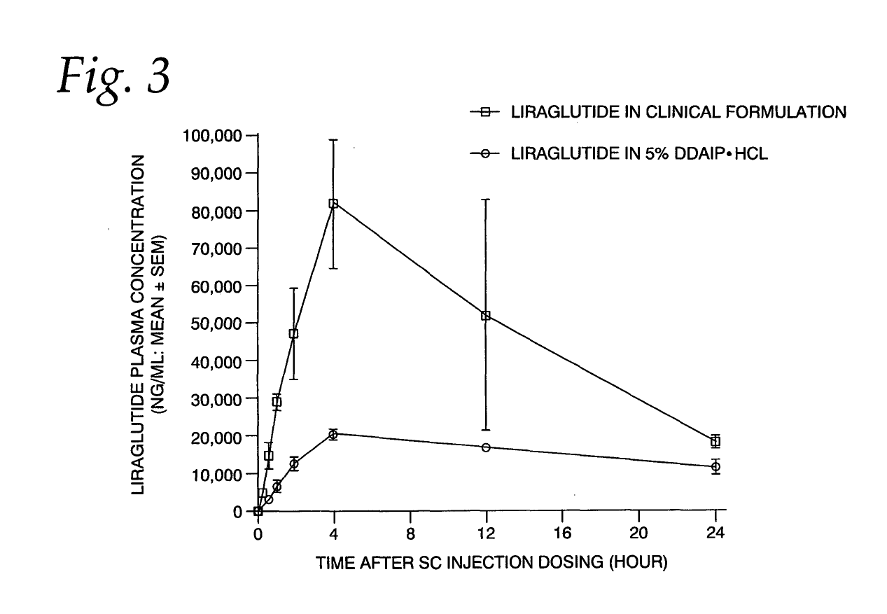

FIG. 3 shows the pharmacokinetic profiles after subcutaneous treatment with liraglutide in Groups 1 and 2. Table II below, details individual pharmacokinetic parameters calculated with PK Solutions 2.0 software (Summit Research Services, Montrose, Colo.). In comparison to treatment with liraglutide in clinical formulation alone, the addition of 5% w/v DDAIP.HCl resulted in lower Cₘₐₓ and AUC values. In contrast, administration with 5% w/v DDAIP.HCl resulted in higher t_{1/2}, Vd and MRT values. In conclusion, this study illustrates that therapeutically-relevant systemic levels of liraglutide can be achieved when dosing with a 5% w/v DDAIP.HCl formulation. Furthermore, such levels can be maintained for a longer period of time than with the clinical formulation (t_{1/2}, MRT) and better distribution to tissues (Vd), thus DDAIP-containing formulations provide a clinical advantage.

TABLE II
Results of the LCMS-MS Analysis of the Samples.

| Formulation | Parameter (units) | |
|---|---|---|
| (Group 1) | Cmax (ng/mL) | 81,560 |
| Liraglutide | Tmax (hr.) | 4.0 |
| (VICTOZA ®) | T½ (hr.) | 15.7 |
| | Vd (mL) | 201 |
| | CL(ml/hr) | 18.1 |
| | AUC (0-24) (ng-hr/mL) | 1,128,000 |
| | AUC (0-∞) (ng- hr/mL) | 1,323,000 |
| | Mean Residence Time (MRT, hr.) | 13.1 |
| (Group 2) | Cmax (ng/mL) | 20,370 |
| Liraglutide | Tmax (hr.) | 4.0 |
| (VICTOZA ®) | T½ (hr.) (extrapolated) | 25.3 |
| with 5% w/v | Vd (mL) | 1,024 |
| DDAIP•HCl | CL (ml/hr) | 36.3 |
| | AUC (0-24) (ng-hr/mL) | 356,900 |
| | AUC (0-∞) (ng- hr/mL) | 661,800 |
| | Mean Residence Time (MRT, hr.) | 30.1 |

### Example 4

### Effects of Insulin in DDAIP.HCl on Blood Glucose Levels

Insulin (2.5 IU/kg) was combined with 0.9% saline and 20% w/v DDAIP.HCl in water (pH 8.5) and was administered subcutaneously (SC dose, 5 ml/kg) to male C57BL/6J mice, (n=6, Jackson Labs., average weight of 33.4 g). For comparison, insulin (2.5 IU/kg) in 0.9% saline without DDAIP.HCl was also administered subcutaneously to mice (n=6). The mice were initially dosed with insulin in the fed state, then food was withdrawn after SC dosing and food was reintroduced to the mice at eight hours after injection. Water was available ad libitum at all times.

Blood glucose levels were measured before administration and were monitored after injection at various time intervals thereafter, using a hand-held glucometer (ACCU-CHEK®, Aviva). Blood samples were obtained from the tail vein initially (T₀), and after dosing at time points of 15 minutes, 30 minutes, 1 hour, 2 hours, 3 hours, 4 hours, 6 hours, 8 hours, 10 hours, and 26 hours.

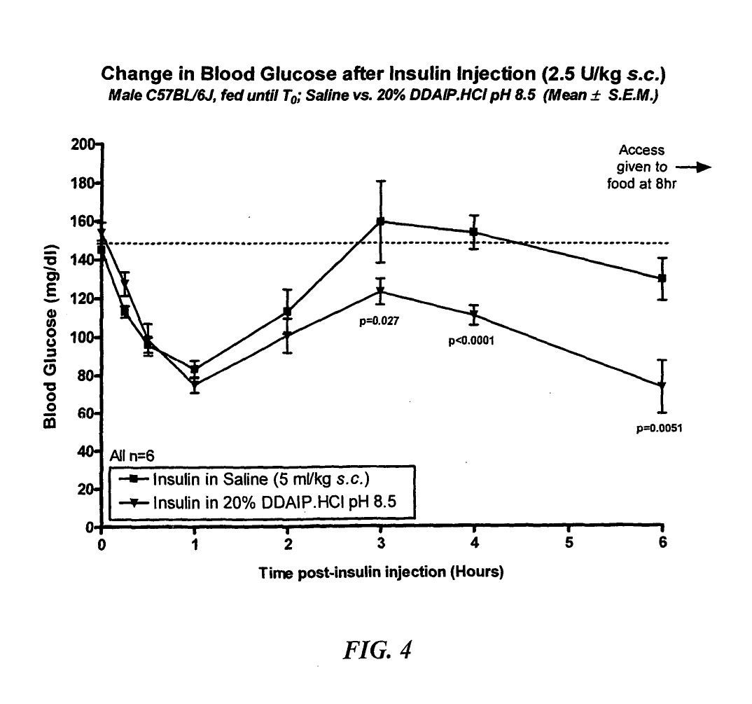

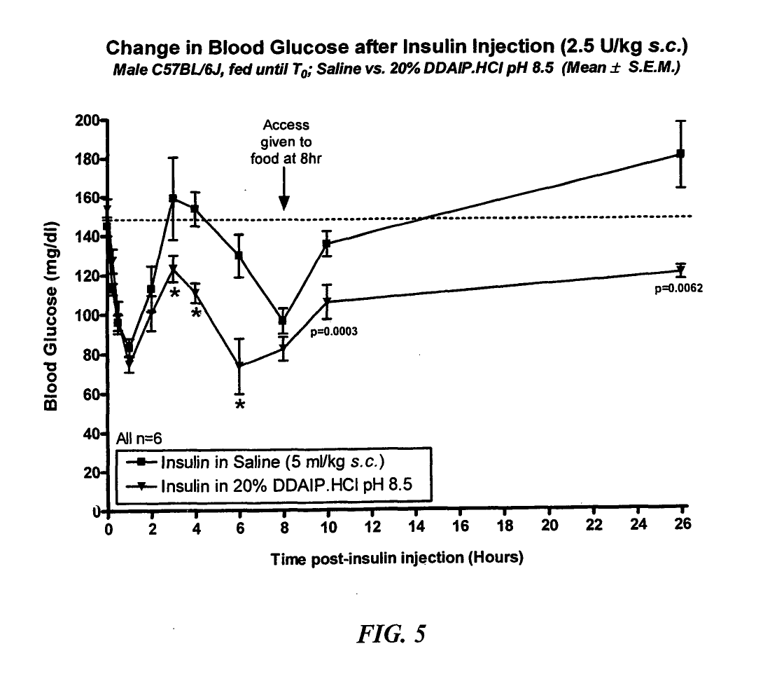

The changes in blood glucose levels are shown in FIG. 4 for mice receiving 2.5 IU/kg insulin subcutaneously over a period of six hours, before being given access to food, and in FIG. 5 for the mice over the entire 26-hour period.

The obtained data presented in FIG. 4 show that over the period of 0-6 hours, the AUC value achieved with insulin in saline was 778±53 and the AUC value achieved with insulin in saline with 20% DDAIP.HCl was 621±29 (at a confidence level of P=0.0256 by two-tailed t-test). The obtained data presented in FIG. 5 show over a period of 0-26 hours, the AUC value achieved with insulin in saline was 3,453±170 and the AUC value achieved with insulin in saline with 20% DDAIP.HCl was 2,567±80 (at a confidence level of P=0.0008 by two-tailed t-test).

The results indicate that the AUC values at 0-6 hours and at 0-24 hours were significantly lower in those mice injected with insulin in saline with 20% w/v DDAIP.HCl (pH8.5) than in mice injected with insulin in saline, without DDAIP.HCl present. The ability of 20% DDAIP.HCl to increase the effectiveness of SC administered insulin was illustrated by the larger decreases in blood glucose levels at individual time points and by the lower AUC values throughout the duration of the study.

Parenteral delivery is preferred for the present compositions. Particularly preferred is subcutaneous delivery to maximize the depot effect.

The present compositions can be formulated as solutions, lyophilized powders, capsules, tablets, liposomes, and the like dosage forms depending on the particular therapeutic peptide and the desired route of administration.

Dosage forms suitable for the therapeutic peptide compositions include parenteral solutions, capsules, tablets, suppositories, gels, creams, and the like.

The foregoing description and the examples are intended as illustrative but are not to be taken as limiting. Still other variations within the spirit and scope of the present invention are possible, and will readily present themselves to those skilled in the art.

1. A composition which comprises a therapeutic peptide, an alkyl N,N-disubstituted amino acetate, and a physiologically acceptable carrier.

2. The composition in accordance with claim 1 wherein the therapeutic peptide is insulin and the alkyl N,N-disubstituted amino acetate is represented by the formula 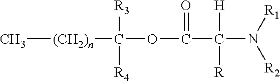
wherein n is an integer having a value in the range of about 4 to about 18; R is a member of the group consisting of hydrogen, C₁ to C₇ alkyl, benzyl and phenyl; R₁ and R₂ are members of the group consisting of hydrogen and C₁ to C₇ alkyl; and R₃ and R₄ are members of the group consisting of hydrogen, methyl and ethyl.

3. The composition in accordance with claim 1 wherein the therapeutic peptide is insulin and the alkyl N,N-disubstituted amino acetate is dodecyl 2-(N,N-dimethylamino) propionate.

4. The composition in accordance with claim 1 wherein the therapeutic peptide is insulin and the alkyl N,N-disubstituted amino acetate is dodecyl 2-(N,N-dimethylamino) propionate hydrochloride.

5. The composition in accordance with claim 1 wherein the therapeutic peptide is a monoclonal antibody.

6. The composition in accordance with claim 5 wherein the monoclonal antibody is Rituximab.

7. The composition in accordance with claim 5 wherein the monoclonal antibody is Raptiva.

8. the composition in accordance with claim 1 wherein the therapeutic peptide is a cytokine and the alkyl N,N-disubstituted amino acetate is dodecyl 2-(N,N-dimethylamino) propionate.

9. The composition in accordance with claim 1 wherein the therapeutic peptide is a cytokine and the alkyl N,N-disubstituted amino acetate is dodecyl 2-(N,N-dimethylamino) propionate hydrochloride.

10. The composition in accordance with claim 9 wherein the cytokine is a human granulocyte colony-stimulating factor (G-CSF).

11. The composition in accordance with claim 9 wherein the cytokine is a granulocyte macrophage colony-stimulating factor (GM-CSF) recombinant.

12. The composition in accordance with claim 1 wherein the therapeutic peptide is selected from the group consisting of a glucagon-like peptide (GLP-1) and analogs thereof and the alkyl N,N-disubstituted amino acetate is dodecyl 2-(N,N-dimethylamino) propionate.

13. The composition in accordance with claim 12 wherein the alkyl N,N-disubstituted amino acetate is dodecyl 2-(N,N-dimethylamino) propionate hydrochloride.

14. The composition in accordance with claim 13 wherein the therapeutic peptide is liraglutide.
